(19) United States
(12) Patent Application Publication
(10) Pub. No.: US 2004/0152186 A1
(43) Pub. Date: Aug. 5, 2004

(54) Culturing apparatus

(76) Inventors: Kazutoshi Kan, Chiyoda (JP); Ikuo Takeuchi, Chiyoda (JP); Shio Miyamoto, Kawasaki (JP)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/767,993

(22) Filed: Feb. 2, 2004

(30) Foreign Application Priority Data
Feb. 4, 2003 (JP) 2003-026577

(51) Int. Cl. C12M 1/36

(52) U.S. Cl. 435/286.6; 435/286.4; 435/303.1

(57) ABSTRACT

A culturing apparatus comprises first and second chambers. The first chamber has air-lock type inlet-outlets in a plural number thereof, and in an inside of which is provided the second chamber for culturing cells therein. Within the first chamber is disposed a manipulator, which is operable through a remote control or an automatic control. The manipulator is able to access both at least one of the air-lock type inlet/outlets and the second chamber. Each of the air-lock type inlet/outlets is divided into two (2) portions by two (2) pieces of doors, and the door of one divided communicates that portion divided to an inside of the chamber. The door of other divided communicates to an outside of the culturing apparatus.

### BACKGROUND OF THE INVENTION

The present invention relates to a culturing apparatus for use of cultivating or culturing vital tissues. In a regenerative medical treatment, etc., for the purpose of medical treatment by culturing the vital tissues, a very small amount of the vital tissues sampled from an organism is cultured up to a certain amount of the vital tissues. An example of those is described, for example, in Patent Document 1 or Patent Document 2. Namely, in Japanese Patent Laying-Open No. Hei 5-292990 (1993), for example, for preventing virus from contamination within the cell culturing apparatus, as well as, from being diffused into an outside of the cell culturing apparatus, the cell culturing apparatus itself is made of a material, which has no permeability against at least the cells and the virus. And, a part of a container or vessel is formed to be a cell growing space, which is built up with a film of porous polymer having permeability with respect to nutriments, bleeding factors, and gases, and also passage spaces for the nutriments, bleeding factors, and gases, being adjacent with the cell growing space through that porous polymer film.

Also, in Japanese Patent Laying-Open No. 2001-238663 (2001), for example, there is described that, for the purpose of achieving effective culture in an outside of a living body while preventing it from the contamination, in the cultivation of cells and/or tissues, a culture chamber is provided under the controlled environment, such as, that imitating a living body, wherein a culture fluid or solution is supplied while maintaining the cells and tissues within this culture chamber.

In the culturing apparatus, which is disclosed in the Japanese Patent Laying-Open No. Hei 5-292990 (1993) mentioned above, though the cells and/or viruses are cultured with using a same vessel (e.g., an integrated vessel) all over the culturing processes thereof, however it is conducted by a hand to plant the cells or the like within the vessel. Also, in the culturing apparatus described in the Japanese Patent Laying-Open No. 2001-238663(2001), it must be conducted by hand, for example, when supplying the cells into the culturing apparatus and/or when exchanging a culture medium. In the apparatus described in this Patent Document 2, further a robot is provided within a clean room, for automating conveyance of a dish and exchange of the culture medium, however a human must enters into an inside of the clean room in other works than those mentioned above.

In the culturing or cells, the human can be a source of contamination at the most. Therefore, conventionally, an area where the human must enter therein is made of a clean room, and the human must wear clean clothes and also pass through an air curtain by a number of times, thereby obtaining dustproof and reducing dusts therein. As a result thereof, facilities thereof come to be large for such the dustproof and dust removing, therefore, an expansive amount of costs is needed for. Also, even if providing strict measures for an antipollution or anti-contamination, it is difficult to remove the source of contamination completely, if a human enters therein.

### BRIEF SUMMARY OF THE INVENTION

An object according to the present invention, being accomplished by taking such the drawbacks of the conventional art mentioned above into the consideration, is to provide a culturing apparatus, being able to be manufactured with a low cost and eliminate the contamination therefrom.

For accomplishing the object mentioned above, according to the present invention, there is provided a culturing apparatus for culturing cellular tissues therein, comprising: a first chamber; a plural number of air-lock type inlet/outlets, being provided in said first chamber; a second chamber for culturing the cells therein; and a manipulator operating within said first chamber, through remote control or an automatic control, wherein said manipulator can access to both, at least one of said air-lock type input/outputs and said second chamber.

And, according to the present invention, in the culturing apparatus as described in the above, preferably, each of said air-lock type inlet/outlets is divided into two (2) portions by two (2) pieces of doors, in which the door of one portion divided communicates that portion to an inside of said first chamber, while the other portion divided communicates to an outside of said culturing apparatus, and preferably, each of said plural number of air-lock type inlet/outlets has check valves on a side surface portion opposing to the inside of said first chamber and on a side surface portion opposing to the outside of said culturing apparatus.

Also, according to the present invention, the culturing apparatus as described in the above, preferably, further comprises a turntable being able to hold an integrated vessel within said second chamber, wherein a door is provided on a side surface or a bottom surface of said chamber, for enabling said manipulator to access to this turntable. More preferably, said turntable is rotatable in a direction of periphery thereof by an angle being equal or greater than 360 degree, and the medium within the integrated vessel is flowable or the position of the integrated vessel is changeable.

And, according to the present invention, the culturing apparatus as described in the above, preferably further comprises a supply source for supplying a medium to the integrated vessel held within said second chamber and a controlled gas to said first chamber, and a control apparatus for controlling said manipulator, and also preferably, further comprises control means for controlling flow, temperature or humidity of gas communicating within said first chamber.

### BRIEF DESCRIPTION OF THE SEVERAL VIEWS OF THE DRAWING

Those and other objects, features and advantages of the present invention will become more readily apparent from the following detailed description when taken in conjunction with the accompanying drawings wherein:

FIG. 1 shows an upper side view of a culturing apparatus, according to one embodiment of the present invention;

FIG. 2 shows a front view of the culturing apparatus, according to the one embodiment of the present invention;

FIG. 3 shows a side view of the culturing apparatus, according to the one embodiment of the present invention;

FIGS. 4(a) and 4(b) show the views of an inner culturing apparatus to be used within the culturing apparatus shown in FIG. 1 mentioned above, and in particular, FIG. 4(a) is an upper view while FIG. 4(b) a side view thereof;

FIG. 5 is a view for explaining the operation of the culturing apparatus mentioned above; and

FIG. 6 is a perspective view of an embodiment of a culturing system, according to the present invention.

### DETAILED DESCRIPTION OF THE INVENTION

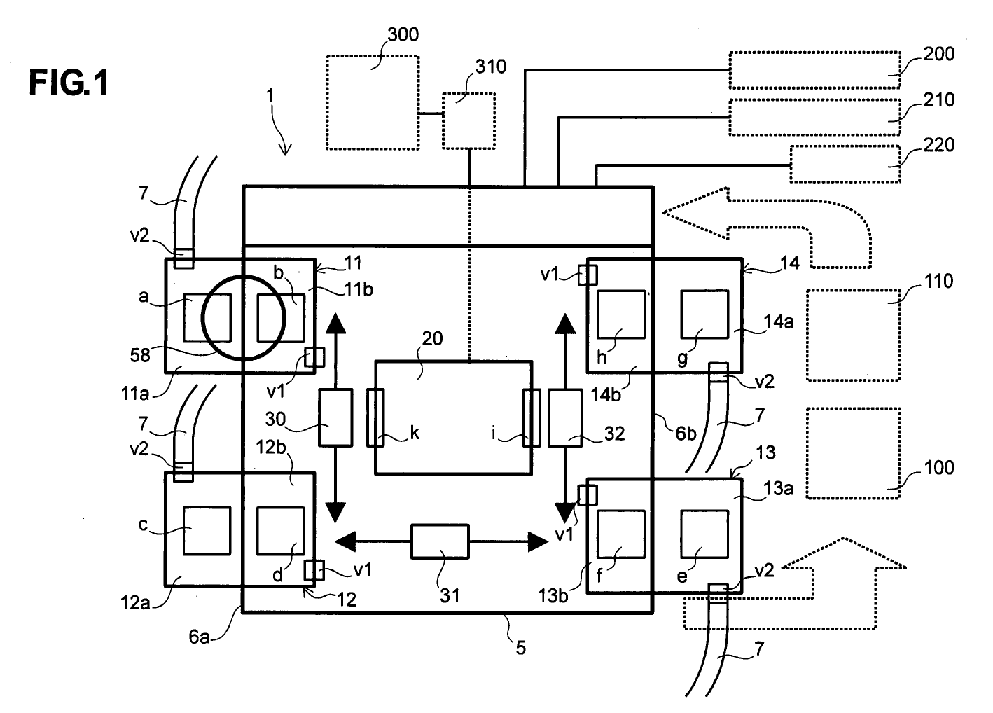

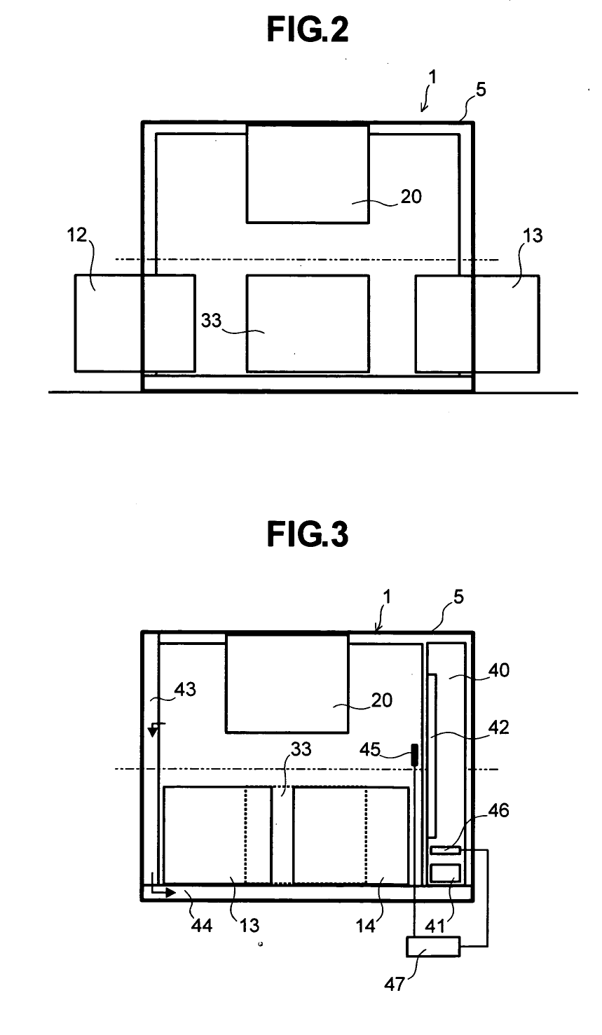

Hereinafter, embodiments according to the present invention will be fully explained by referring to the attached drawings. Those FIGS. 1 to 3 show the culturing apparatus, according to one embodiment of the present invention, and in particular, FIG. 1 shows an upper view, FIG. 2 a front view, and FIG. 3 a side view thereof. The culturing apparatus 1 comprises a rectangular chamber 5, and a plural number of air-lock portions 11-14, which are attached on both side surfaces 6a and 6b of the chamber 5 opposing to each other. The air-lock portions 11-14 includes an air-lock portion 11 for use of introducing or inputting cells to be cultured therein, an air-lock portion 12 for use of inputting therein detachable tip portions and/or tubes, etc., of a disposable pipette, which is provided within the chamber 5 for use of suction and/or dividing injection, an air-lock portion 13 for use of outputting or taking-out the used tip portions and/or tubes of the pipette therefrom, and an air-lock portion 14 for use of inputting and/or taking-out of a cell culture cartridge 50.

The cell culture cartridge 50 is a kind of an integrated container or vessel for culturing cells therein. The integrated vessel is produced for the purpose to be used, continuously, over all of the processes of culturing. On the integrated vessel is formed an opening portion, through which a medium is introduced therein, and an opening portion, through which the medium is disposed of, and it is preferably made of a resin, an inner surface or an outer surface of which is adhered with a film, or a glass. Also, it may be processed so be suitable for culturing in an inside thereof.

Each of the air-lock portions 11-14 is built up with two (2) chambers 11a and 11b, and each of those chambers 11a and 11b is provided with a door a-h. In each of the air-lock portions 11-14, there is provided a check valve v1 for enabling a gas to pass through from an inside of the chamber 5 into an inside of the air-lock portion 11-14, but cut off a flow from an inside of the chamber 5 into an inside of each of the air-lock portions 11-14 is provided on a side surface of the air-lock portion 11-14 within an inside of the chamber 5. On an outside of the chamber 5 of each of the air-lock portions 11-14, a tube 7 is connected, which can perform suction. Between the tube 7 and the air-lock portion 11-14, there is provided a check valve v2, for enabling a gas to pass through from an inside of the air-lock portion to an outside of the culturing apparatus 1, but preventing a flow from flowing in a reverse direction.

Normally, an inside of the culturing apparatus 1 is kept to be positive in pressure, comparing to that of an outside. The pressure of the tube 7 connected to the air-lock portions 11-14 is kept to be equal to an internal pressure in the culturing apparatus 1. When opening any one of an outside doors “a”, “e” and “h” of those air-lock portion 11, 12, 14 under such the condition, an air inside the chamber 5, passing through the check valve v1 of the inner side, flows out from the doors “a”, “e” and “h” which are opened. Then, the materials or the like are positioned within an inside of the air-lock portions 11, 12 and 14, which are necessary for culturing and should be supplied into the culturing apparatus 1.

Next, while closing the doors “a”, “e” and “h”, the pressure within the tube 7 is reduced for predetermined time duration with using a vacuum pump, etc., not shown in the figure. The gas, such as, containing therein an air, carbon dioxide, oxygen, etc., for example, within the air-lock portions 11, 12 and 14, flows passing through the check valve v2 into the tube 7, thereby ventilating an inside of the vessel 5. Since it is possible to supply the materials or the like into an inside of the chamber 5 without assistance of a human hand, in this manner, therefore it is possible to eliminate an ill influence of the human hand, in particular, when supplying such the materials or the like into the culturing apparatus 1.

When completing the ventilation, the pressure within the tube 7 is brought to be equal that within the chamber 5. This stops the air from flowing out, from the inside of the chamber 5. After treating the necessary processes within the air-lock portions 11, 12 and 14, then the doors “b”, “d” and “g” of an inside of the chamber 5 are opened. The materials or the like are taken out from the air-lock portions 11, 12 and 14, with using manipulators 30-32 disposed within the chamber 5.

When taking out those that are used or spent in the processes of culturing from the inside of the chamber 5, the inside doors “f” and “h” of the chamber 5 are opened, for use of taking-out. Those, which should be taken out from the chamber 5, are positioned within the air-lock portions 13 and 14, with using the manipulators 30-32. Thereafter, the inside doors “f” and “h” of the chamber 5 are closed, and then, the necessary processes are treated within the air-lock portions 13 and 14 on those, which should be taken out. The doors “e” and “g” are opened, communicating to an outside of the air-lock portions 13 and 14. Under this condition, since the pressure within the air-lock portions is positive, no air flows into from an outside even if opening the outside doors “e” and “g”.

After taking out the waste matter or the like to be discharged from the air-lock portions 13 and 14, by a hand or with using the manipulator or the like, not shown in the figure, the outside doors “e” and “g” of the air-lock portions 13 and 14 are closed. After closing up the outside doors “e” and “g”, the pressure on the side of the tube 7 is reduced down by means of the vacuum pump, etc. The gas within the air-lock portions 13 and 14 are discharged into an outside through the tube 7, thereby ventilating. After the ventilation, the pressure within the tube 7 is increased, again, thereby stopping the gas from flowing. According to the present embodiment, it is possible to conduct supply and taking-out of those, which are used in the culturing processes, into/from the calculating apparatus 1, but with the minimal degree of contamination.

Further, the details of operations of the culturing of cells are as below. The cells to be cultured are put into the air-lock portion 11, to be centrifugalized with using a centrifugal separator 58, which is disposed within an inside of the air-lock portion 11. After completing the centrifugation, the inside door “b” is opened and cleaning is conducted by means of the medium. A portion, being collected by the centrifugation, i.e., that containing the cells therein, as a target of the culturing, is sucked with using a suction means not shown in the figure, attached at a tip of the manipulator 30. The cells sucked are injected into a cartridge 50, being the integrated vessel mentioned above, from an inlet thereof, which is not shown in the figure but formed thereon. With this, the cells are planted within the cartridge 50. Those series operations are conducted by the manipulator 30, which has the function of a pipette. For controlling of the manipulator 30, a driver 33 is utilized, as well as, using an automatic control developed in a robot technology.

The tip of the pipette is exchanged in the following manner. After operating the doors “c” and “d” of the air-lock portion 12 for use of inputting of the pipette as was mentioned above, the pipette tip is positioned in the air-lock portion 12 for use of inputting of the pipette. Then, with operating the doors “c” and “d” of the air-lock portion 12, the air-lock portion 12 is brought into the condition, so that the manipulator 30 having the pipette function can be operated. The detachable pipette tip is attached onto a tip of the manipulator 30 through the automatic control or a remote control. After completing the use of the pipette, the pipette tip is removed from the tip of the manipulator 30 with using the manipulator 31 having a holding function. After operating the doors “c” and “d” of the air-lock portion 12, the pipette tip is taken out from the air-lock portion 13 for use of discharge, by using a hand or a manipulator.

The operation of supplying the cartridge 50 for culturing into an inside of the chamber 5 or taking it out from the inside of the chamber 5 is as below. After operating the doors “g” and “h” of the air-lock portion 14 for use of inputting and taking-out of the integrated vessel as was mentioned above, the cartridge 50 is put into the air-lock portion 14, which is sealed after being sterilized. While partitioning within an inside of the air-lock portion 14 by operating the doors “g” and “h”, the seal is broken out by a seal removing means not shown in the figure. This can be executed easily; such as, a cutter provided within the air-lock portion 14. Also, if it is a packing vessel made of stick film, for example, it is possible to break the seal of the cartridge 50 without an assistance of a hand; such as, by pulling up the seal opening by the tip portions of two (2) pieces of manipulators, or by using a jig for pulling the seal opening into the opposite directions with each other.

With operating the door “h”, the manipulator 32, which is disposed within the chamber 5 and has the holding function, is brought into the condition, so that it can access into the air-lock portion 14b. The manipulator 32 holds the cartridge 50 positioned in the air-lock portion 14b, and it disposes the cartridge 50 within an inside of the inner culturing apparatus 20 provided in an upper portion within the chamber 5. In the inside of the inner culturing apparatus 20, there is provided a supporting means for supporting the cartridge 50 rotatable.

On two (2) side surfaces of the inner culturing apparatus 20 opposing to each other, there are provided doors “i” and “k”. After opening the door “i” locating on a side of the manipulator 32, the manipulator 32 rotates the empty cartridge 50 in an inside of the inner culturing apparatus 20. Then, the door “k” is opened, which is located on the side of the manipulator 30 having the pipette function, and then the manipulator 30 plants the cells onto the empty cartridge 50. Thus, into an injection inlet 50d formed on an end surface of the cartridge 50, the planting is conducted by means of a thin hollowed needle, which is formed at the pipette tip. After completing the planting, the tube for use of supplying the medium and the tube for use of discharging the medium are connected with each other, by using the manipulator 30. The manipulators 30 and 32 are moved out from the inner culturing apparatus 20, thereby closing up the doors “i” and “k”, and then the culture is started within the inner culturing apparatus 20.

Into the inner culturing apparatus is supplied the medium, being heated and adjusted to an appropriate temperature, from a medium supply source 300, which is provided in an outside of the culturing apparatus 1, by means of a pump 310. The gas within an inside of the chamber 5 is supplied from an oxygen supply source 200, a carbon dioxide supply source 210, and/or an air supply source 220, etc., which are provided in an outside thereof. Into an inside of the chamber is formed a filter portion 40, thereby removing dusts contained within the gas, which is/are supplied from the outside gas supply sources 200-220. Also, in an inside of the discharge passage, there are provided a sensor 100 for measuring the contents of the gas discharged from the culturing apparatus and a temperature sensor 110.

As is shown in detail in FIG. 3, on the bottom portion of the filter portion 40 is provided a fan 41. Also, at a middle position of the filter portion 40 in the vertical direction thereof is attached a dustproof filter 42. The gas passing through the dustproof filter 42, after passing through an inside of the chamber 5, passes through an opposing wall 43 being formed in a matrix manner. Then, passing through a grazing portion 44, which is formed on the bottom portion within an inside of the bottom chamber 5, it flows in the horizontal direction, and then it is turned back to the fan 41.

A temperature sensor 45 is provided within an inside of the chamber 5 for detecting the temperature of the gas passing through the inside of the chamber 5. Upon the basis of the gas temperature detected by this temperature sensor 45, a temperature controlling apparatus 47 controls a heater 46 provided in the vicinity of the fan 41, thereby to heat up the gas to be a predetermined value in temperature, or increases an amount of air to be supplied from the outer air supply source 220. However, the outer air supply source 220 is kept to be low in temperature, therefore it is used for lowering the gas temperature. It is also possible to bring the gas into a predetermined composition, while measuring the composition of the gas with using a measurement means not shown in the figure, by supplying the necessary gasses from the outer gas supply means 200-220.

According to the present embodiment mentioned above, since the manipulator is controlled in accordance with a predetermined steps, after supplying the original cells to be cultured, the pipette tip portion, and the cartridge, respectively, from the air-lock portions 11, 12 and 14 into the inside of the chamber 5, it is possible to plant the cells in an inside of the cartridge without assistance of a hand. However, upon culturing, the cells are cultured by exchanging the medium flowing, appropriately.

The steps for taking out the cartridge, being filled up with the cultured cells after completing the culturing thereof, are as below. The manipulator 32 having the holding function opens the door “i” of the inner culturing apparatus 20, and it removes the tube for use of supplying the medium and the tube for use of discharging from the cartridge 50. And, it takes out the cartridge 50 from the inner culturing apparatus 20 through the door “i”.

By operating the door “h” of the air-lock portion 14 for use of inputting and taking-out of the cartridge, the manipulator 32 is brought into the condition, so that it can access to the air-lock portion 14. Since the manipulator 32 is able to access to the air-lock portion 14, then the manipulator 32 puts the cartridge 50 in the air-lock portion 14. Thereafter, the door “h” is closed, and the door “g” is opened, so that the cartridge 50 can be accessed from an outside.

On the other hand, for the purpose of taking out the tubes removed from, the door “f” of the air-lock portion 13 for use of discharging the disposals, thereby bringing the manipulator 32 to be able to access the air-lock portion 13. The manipulator 32 puts the tubes removed from in the air-lock portion 13. Thereafter, the doors “e” and “f” are operated, so that it is possible to access from an outside of the chamber 5 to the air-lock portion 13. A human being who wears sterilized groves or a manipulator for use of taking-out accesses the air-lock portion 13, thereby taking out the tubes into an outside, for disposal thereof.

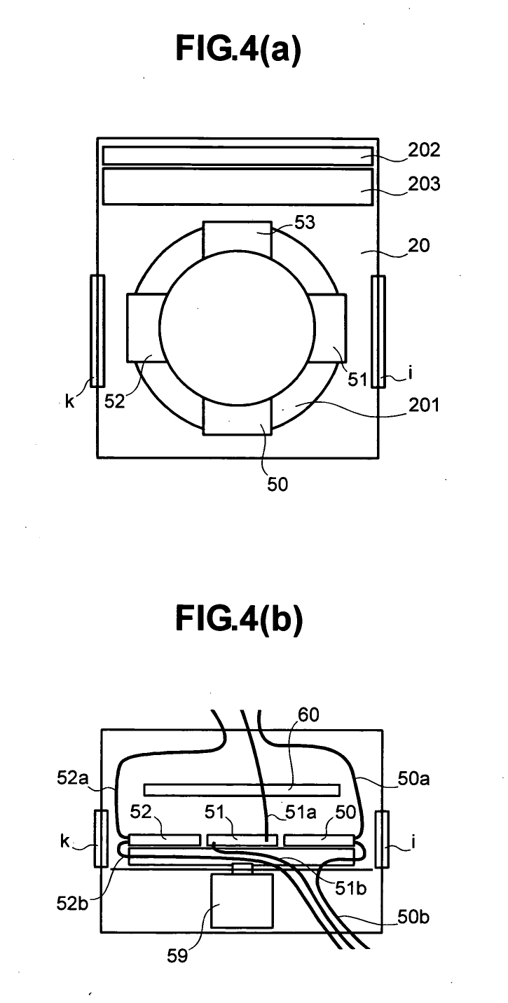

Details of the inner culturing apparatus 20 will be explained by referring to FIGS. 4(a) and 4(b). Herein, FIG. 4(a) is an upper view of an embodiment of the inner culturing apparatus 20, and FIG. 4(b) a side view thereof. On one side surface of the inside of the inner culturing apparatus 20 is provided a solid temperature controller 203 through a fan 202. The fan 202 diffuses or spreads the gas within the inner culturing apparatus 20 weakly. The solid temperature controller 203 is made of, such as, a Peltier element being able to absorb and generate heat, for example, thereby controlling the temperature within the inner culturing apparatus 20.

Around a central portion of the inner culturing apparatus 20, there is provided a turntable 201, which is driven by a servomotor 59. On the turntable 201 can be mounted the cartridge 50 at an equal angle in the peripheral direction thereof. In this FIG. 4, the cartridges 50-53 are disposed equally in the peripheral direction thereof. Those cartridges 50-53 are detachable on the turntable 201 mechanically, electro-statistically, or electro-magnetically. The turntable 201 is so constructed that it can rotate by one round or more than that. For this reason, when the manipulator positions the cartridges 50-53 on the turntable 201 passing through the door “i” on a side of the manipulator 32, it can dispose them at any position on the turntable 201. Above the turntable 201, a tube guide 60 is provided. To the cartridges 50-53 are connected the tubes 50a-53a for use of supplying the medium and the tubes 50b-53b for use of discharging the medium, respectively. The tube guide 60 makes the medium supplying tubes 50a-53a rotate along with the periphery while guiding them, when the turntable 201 rotates.

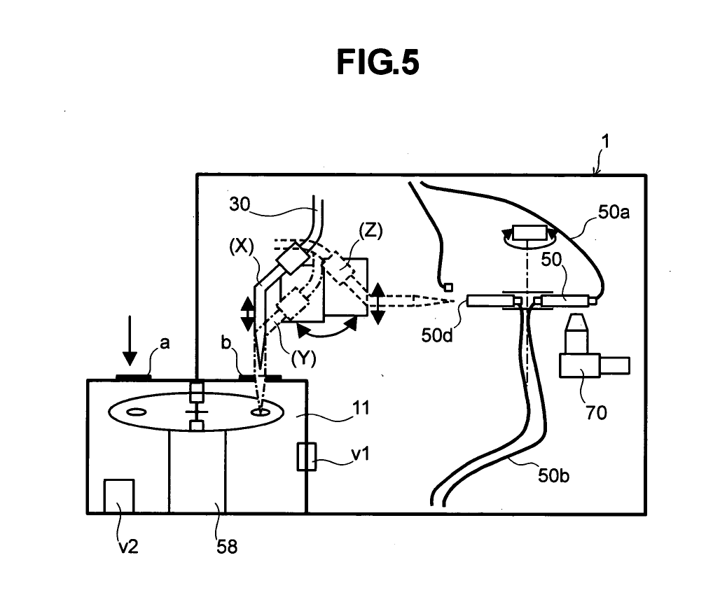

Explanation will be given about detailed operation of the pipette operation with using the manipulator having the pipette function, by referring to FIG. 5. The manipulator 30 is able to move vertically and to rotate, and to move horizontally, and also in the direction orthogonal to the sheet surface of the drawing, though not shown in the figure. With the movements into those directions, the manipulator 30 can access to the air-lock portion 11 from the door portion b, thereby sucking the cells of the target, which is separated by the centrifugal separator 58. After being attenuated, the sucked cells are planted into the cartridge 50 from the injection inlet 50d. Namely, the manipulator 30 changes the position thereof from the condition (x) of being indicated by solid lines into that (y) of being indicated by one-dotted chain lines, and then into the condition (z) indicated by broken lines. With this, it is possible to plant the cells into the integrated vessel in an unmanned manner, by using the manipulator. Also, the cells are loosened depending upon the necessity thereof, with using other vessel(s) within the centrifugal separator 58. Within the inside of the inner culturing apparatus 20 are provided a television camera and an illumination system 70. This television camera optically observes the cultured cells under the cultivation thereof, thereby observing anything unusual thereof. Since the cartridge 50 is made up with a transparent vessel, it is possible to monitor the condition of the cells by means of the television camera.

The culturing apparatus according to the present embodiment has a dual structure, in which the inner culturing apparatus 20 is provided within the chamber 5. With this, it is possible to reduce the contamination, greatly. Also, there is/are provided the air-lock portion(s) for each of various purposes thereof, so as to enable controlling on pressure of each of the air-lock portions by means of the check valves and the suction means connected to those check valves, therefore it is possible to reduce the contamination that may occurs when supplying and/or discharging the necessary cells and/or materials from/into an outside, greatly.

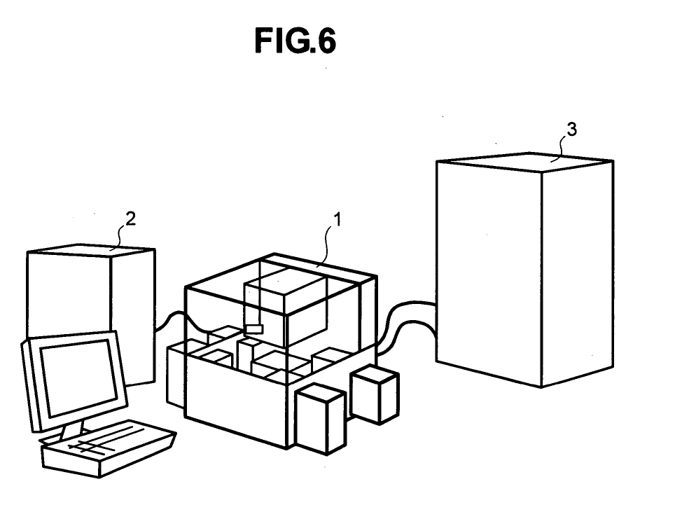

FIG. 6 shows an example of the culturing system comprising the culturing apparatus, which was mentioned in the above embodiment. The present system comprises an auxiliary or accessory portion 3, building the medium supply source and the gas supply source therein, the culturing apparatus 1, and a culturing control apparatus 2 for controlling the culturing apparatus. Thus, the culturing of cells is controlled with using programs, which are memorized in the control apparatus 2 in advance.

In the embodiment mentioned above, the operation of the manipulator automated, completely, however it is also possible to provide a camera on the manipulator, thereby enabling the remote supervisory operation. In this instance, it is possible to operate the manipulator with high accuracy, much more.

According to the present invention, since the integrated vessel introduced into from the air-lock portion is operated unmanned manner within the chamber, it is possible to exclude or eliminate the human being that can be the most possible source of contamination from the culturing equipment, thereby enabling the culturing of cells, cleanly, but without a large-scaled facilities.

The present invention may be embodied in other specific forms without departing from the spirit or essential feature or characteristics thereof. The present embodiment(s) is/are therefore to be considered in all respects as illustrative and not restrictive, the scope of the invention being indicated by the appended claims rather than by the forgoing description and range of equivalency of the claims are therefore to be embraces therein.

What is claimed is:

1. A culturing apparatus for culturing cellular tissues therein, comprising:
a first chamber;
a plural number of air-lock type inlet/outlets, being provided in said first chamber;
a second chamber for culturing the cells therein; and
a manipulator operating within said first chamber, through remote control or an automatic control, wherein said manipulator can access to both, at least one of said air-lock type input/outputs and said second chamber.

2. A culturing apparatus, as described in the claim 1, wherein each of said air-lock type inlet/outlets is divided into two (2) portions by two (2) pieces of doors, in which the door of one portion divided communicates that portion to an inside of said first chamber, while the other portion divided communicates to an outside of said culturing apparatus.

3. A culturing apparatus, as described in the claim 2, wherein each of said plural number of air-lock type inlet/outlets has check valves on a side surface portion opposing to the inside of said first chamber and on a side surface portion opposing to the outside of said culturing apparatus.

4. A culturing apparatus, as described in the claim 1, further comprising a turntable being able to hold an integrated vessel within said second chamber, wherein a door is provided on a side surface or a bottom surface of said chamber, for enabling said manipulator to access to this turntable.

5. A culturing apparatus, as described in the claim 1, further comprising a supply source for supplying a medium to the integrated vessel held within said second chamber and a controlled gas to said first chamber, and a control apparatus for controlling said manipulator.

6. A culturing apparatus, as described in the claim 1, further comprising control means for controlling flow, temperature or humidity of gas communicating within said first chamber.

7. A culturing apparatus, as described in the claim 4, wherein said turntable is rotatable in a direction of periphery thereof by an angle being equal or greater than 360 degree, and the medium within the integrated vessel is flowable or the position of the integrated vessel is changeable.

8. A culturing apparatus for culturing cellular tissues therein, comprising:
a first chamber;
a plural number of air-lock type inlet/outlets, being provided in said first chamber; and
a second chamber for culturing the cells therein, being provided in said first chamber, wherein each of said air-lock type inlet/outlets is divided into two parts by two (2) pieces of doors, and the door of one of those parts which are divided is provided to be communicate that divided part with an inside of said first chamber, while the door of the other of the parts which are divided is to be communicate with a connection electrode to an outside of the culturing apparatus.

9. A culturing apparatus, as described in the claim 8, wherein each of said plural number of air-lock type inlet/outlets comprises:
check valves, being provide on a partition wall opposing to an inside of said first chamber and a partition wall opposing to an outside of said culturing apparatus, and wherein:
pressure within an inside of said first chamber is set to be higher than that within the inside of said of air-lock type inlet/outlet and the outside of the culturing apparatus, so that when the door of said air-lock type inlet/outlet communicating to the outside of the culturing apparatus is opened, an air flows directing from the inside of the culturing apparatus into the outside thereof through the check valve on the partition wall opposing to the inside of said first chamber, while the air stops when closing the door communicating to the outside of the culturing apparatus; and
next under this condition, before opening the door communicating to the inside of the culturing apparatus, the air is exhausted through the check valve provided on the partition wall opposing to the outside of the culturing apparatus, and thereafter the door communicating to the outside of the culturing apparatus is opened.
